(19) United States
(12) Patent Application Publication
(10) Pub. No.: US 2024/0066108 A1
(43) Pub. Date: Feb. 29, 2024

(54) OVARIAN CANCER VACCINE

(71) Applicants: The Regents of the University of California, Oakland, CA (US); Trustees of Dartmouth College, Hanover, NH (US)

(72) Inventors: Nicole F. Steinmetz, La Jolla, CA (US); Steven Fiering, Hanover, NH (US); Courtney T. Stump, Hanover, NH (US)

(21) Appl. No.: 18/260,741

(22) PCT Filed: Jan. 7, 2022
(86) PCT No.: PCT/US2022/011706

§ 371 Date: Jul. 7, 2023

Related U.S. Application Data
(60) Provisional application No. 63/135,406, filed on Jan. 8, 2021.

(51) Int. Cl. A61K 39/00; A61K 35/17

(52) U.S. Cl. CPC A61K 39/0011; A61K 35/17; A61K 39/4611; A61K 2039/55594; A61K 2039/572; A61K 2239/59

(57) ABSTRACT

Provided are compositions, methods and uses relating to an adjuvant which comprises, or consists essentially of, or consists of a cowpea mosaic virus (CPMV) particle and its combination with one or more antigens or cells, such as an irradiated cancer cell comprising the one or more antigens.

### CROSS-REFERENCE TO RELATED PATENT APPLICATION

This application claims the benefit under 35 U.S.C. § 119(e) of U.S. Provisional Application Ser. No. 63/135,406, filed Jan. 8, 2021, the contents of which are hereby incorporated by reference in its entirety.

### STATEMENT OF GOVERNMENT SUPPORT

This invention was made with government support under Grant Nos. CA218292, CA224605, and CA253615 awarded by the National Institutes of Health (NIH). The government has certain rights in the invention.

### BACKGROUND

Throughout this disclosure reference is made to technical and patent literature to more fully describe the state of the art by an identifying citation or an Arabic numeral. The full bibliographic citation for the references identified by an Arabic numeral is provided immediately preceding the claims.

A serous ovarian carcinoma diagnosis carries a dismal prognosis. Due to the cancer's nonspecific clinical symptoms, the majority of patients are diagnosed with stage III or stage IV disease, among which the five-year survival rates are 42% and 26%, respectively [1]. The current standard of care includes surgical debulking, during which large quantities of tumor are removed from the peritoneal cavity. Surgery is generally followed by carboplatin and paclitaxel chemotherapy and, while most patients enter remission, many later relapse with chemo-resistant tumors [2]. Following relapse, most patients succumb to their disease. Accordingly, there is a significant need for therapies that could be applied during remission to prevent relapse when the tumor burden is very low. This disclosure satisfies this need and provides related advantages as well.

### SUMMARY OF THE DISCLOSURE

Cancer immunotherapies have revolutionized clinical oncology, particularly in the treatment of certain cancers, such as melanoma. However, traditional immunotherapies, such as immune checkpoint blockades, have proven largely unsuccessful in treating ovarian cancer [3-6]. This is likely due to the fact that the ovarian tumor microenvironment is strongly immunosuppressive [7]. While immune checkpoint blockade therapies rely upon revitalizing an existing T cell response, the intense immunosuppression provided by the abundance of M2-type tumor-associated macrophages (TAMs), regulatory T cells (Tregs), and myeloid-derived suppressor cells (MDSCs) in the ovarian cancer microenvironment generally prevents a robust and effective T cell response [8-13].

Immune checkpoint blockade therapies have not successfully treated ovarian cancer, and the field is actively investigating other ovarian cancer immunotherapies, including vaccines [14]. An autologous dendritic cell vaccine against mucin 1 (MUC-1), a tumor-associated antigen (TAA), had promising results in phase II clinical trials [15]. Another promising approach relevant to this study used hypochlorous acid-oxidized ovarian whole tumor lysates to treat dendritic cells, ultimately inducing an anti-tumor CD8+ T cell response and extending survival outcomes [16]. Peptide vaccines, such as those directed against another TAA, NY-ESO-1, have shown promise [17-19]. Peptide vaccines, or antibodies targeting another TAA relevant to ovarian cancer, sperm surface protein 17 (Sp17), have been studied in mice and humans [20-22]. Further, PANVAC is a therapeutic poxviral vaccine containing the genes for the tumor-associated antigens MUC-1 and carcinoembryonic antigen (CEA), as well as immunostimulatory genes CD80, intracellular adhesion molecule-1 (ICAM1), and leukocyte function-associated antigen-3 (LFA3) [23]. It was recently shown that the prophylactic injection of freeze-thawed lysates of a murine ovarian cancer stem-like cell expressing high levels of ROR-1 increased mouse survival [24]. Another study indicated that the prophylactic injection of a TAA, Sp17, and CpG oligodeoxynucleotide, a toll-like receptor (TLR) 9 agonist, dramatically extended survival in mice [25].

Although moderately efficacious, current vaccines are therapeutic and designed to treat active disease. Provided herein are vaccines designed to be delivered during remission, when disease is clinically undetectable, an approach that termed “remission-stage vaccines” that can be modeled with prophylactic vaccines.

Provided herein is a vaccine such as an ovarian cancer vaccine, for example, that uses a novel mix of Cowpea Mosaic Virus (CPMV) (or CPMV particle as referred to herein) mixed with killed cells, as well as related compositions and methods. In some embodiments, the ovarian cancer is ovarian serous carcinoma. In some embodiments, the disclosure relates to therapy and prophylaxis of disease such as ovarian cancer, e.g. ovarian serous carcinoma.

Also provided is a novel plant virus based nanoparticle adjuvant. As used herein, the term adjuvant intends a plant virus based nanoparticle that is mixed with an inactivated or killed ovarian cancer cells (e.g., ovarian serous carcinoma) to prime anti-tumor immunity upon administration to, or immunization of the patient.

In one aspect, provided is a method for inhibiting, delaying, slowing down, or preventing relapse of cancer in a subject in need thereof. The method comprises, consists essentially of, or consist of administering to the subject: (a) an adjuvant which comprises, or consists essentially of, or consists of a cowpea mosaic virus (CPMV) particle, for example, an effective amount of the adjuvant; and (b) one or more cancer antigen(s) or an cancer cell comprising the one or more cancer antigen(s), for example, an effective amount of the cancer antigen(s) or ovarian cancer cell, thereby delaying, slowing down, or preventing the relapse of the cancer in the subject. In some embodiments, the cancer is an ovarian or an ovarian serous carcinoma. Additionally or alternatively, the cell comprising the one or more cancer antigen(s) is an irradiated cancer cell. In one aspect, the cowpea mosaic virus (CPMV) particle is the only active agent in the adjuvant.

In another aspect, provided is a method for treating cancer in a subject in need thereof. The method comprises, consists essentially of, or consist of administering to the subject: (a) an adjuvant which comprises, or consists essentially of, or consists of a cowpea mosaic virus (CPMV) particle, for example, an effective amount of the adjuvant; and (b) one or more cancer antigen(s) or an cancer cell comprising the one or more cancer antigen(s), for example, an effective amount of the antigen(s) or ovarian cancer cell, thereby treating the cancer in the subject. In some embodiments, the cancer is ovarian cancer or ovarian serous carcinoma. Additionally or alternatively, the cell such as the ovarian cancer cell comprising the one or more antigen(s) is an irradiated cell, e.g., an ovarian cancer cell. In one aspect, the cowpea mosaic virus (CPMV) particle is the only active agent in the adjuvant.

In yet another aspect, provided is a method for triggering or enhancing one or more of the following in a subject in need thereof: an immune response, a T cell-dependent immune response, innate immune responses, a T cell priming, a CD8+ T cell-dependent immune response, or an immunological memory. The method comprises, consists essentially of, or consist of administering to the subject (a) an adjuvant which comprises, or consists essentially of, or consists of a cowpea mosaic virus (CPMV) particle, for example, an effective amount of the adjuvant; and (b) one or more antigen(s), or a cell comprising the one or more antigen(s), for example, an effective amount of the antigen(s) or cancer cell, thereby triggering or enhancing one or more of the following in the subject: an immune response, a T cell-dependent immune response, a T cell priming, a CD8+ T cell-dependent immune response, or an immunological memory. In some embodiments, the immune response, T cell priming, or immunological memory is anti-cancer. Additionally or alternatively, the antigen(s) is a cancer antigen(s). In further embodiments, the cell comprising the one or more antigen(s) is a cancer cell. In some embodiments, the cancer is an ovarian cancer such as ovarian serous carcinoma. Additionally or alternatively, the cell comprising the one or more antigen(s) is an irradiated cancer cell, e.g., an ovarian cancer cell or an ovarian serous carcinoma cell. In one aspect, the cowpea mosaic virus (CPMV) particle is the only active agent in the adjuvant.

In one aspect, provided is a kit for use in a method as disclosed herein. The method comprises, consists essentially of, or consists of (a) an adjuvant which comprises, or consists essentially of, or consists of a cowpea mosaic virus (CPMV) particle; and an optional instruction for use. In one aspect, the cowpea mosaic virus (CPMV) particle is the only active agent in the adjuvant. In some embodiments, the kit further comprises (b) one or more antigen(s), or a cell comprising the one or more antigen(s). In some embodiments, the antigen(s) is a cancer antigen(s). In further embodiments, the cell comprising the one or more antigen(s) is a cancer cell. In some embodiments, the cancer is an ovarian cancer or ovarian serous carcinoma. Additionally or alternatively, the cancer cell comprising the one or more antigen(s) is an irradiated cancer cell.

In another aspect, provided is a vaccine composition comprising, consisting essentially of, or consisting of (a) an adjuvant which comprises, or consists essentially of, or consists of a cowpea mosaic virus (CPMV) particle; and (b) one or more antigen(s) or a cell comprising the one or more antigen(s). In some embodiments, the antigen(s) is a cancer antigen(s). In further embodiments, the cell comprising the one or more antigen(s) is a cancer cell. In some embodiments, the cancer is an ovarian cancer or an ovarian serous carcinoma cell. Additionally or alternatively, the cell comprising the one or more antigen(s) is an irradiated cancer cell such as an irradiated ovarian cancer cell or an ovarian serous carcinoma cell. In one aspect, the cowpea mosaic virus (CPMV) particle is the only active agent in the adjuvant.

### BRIEF DESCRIPTION OF THE DRAWINGS

FIGS. 1A-1E: The combination of cowpea mosaic virus nanoparticles (CPMV) and irradiated cells significantly extends survival in a mouse model of ovarian cancer. ID8/VEGFA/defb29 cells were inactivated for intraperitoneal (IP) vaccine injection in one of four different ways: irradiation, freeze-thaw, heat shock, or HOCl oxidation (see methods for cell preparation). Following two vaccinations seven days apart, mice were challenged with live tumor cells and survival tracked. (FIG. 1A) Irradiated p=0.007 compared to freeze-thaw, n=4 in all groups; (FIG. 1B) cells co-delivered IP with 100 μg MPLA. Irradiated p=0.35 compared to freeze-thaw and p=0.59 compared to vehicle, n=4 in all groups except freeze-thaw+MPLA where n=3; (FIG. 1C) inactivated cells were co-delivered IP with 100 μg CPMV, n=4 in all groups, freeze-thaw p=0.03, irradiated p=0.03 compared to vehicle; (FIG. 1D) mice received irradiated ID8/VEGFA/defb29 cells co-delivered IP with PBS, 100 μg CPMV, 100 μg MPLA, or 250 μg DMXAA. n=4 in all groups except irradiated+DMXAA where n=8. Irradiated+CPMV p=0.03 or less when compared to any other group; (FIG. 1E) n=4 in all groups. Irradiated+CPMV p=0.007 or less compared to any other group. (FIGS. 1A-1E) When twice the average length of the survival of vehicle-treated mice had passed, surviving mice were rechallenged with 5×10₆ cells, as denoted by the arrows. p values compare survival curves with a log-rank (Mantel-Cox) test. All p values are compared to vehicle-treated controls unless otherwise noted ** 0.001<p<0.01; * 0.01<p<0.05.

FIGS. 2A-2B: The prophylactic codelivery of irradiated cells and CPMV provides robust, long-term protection from ID8/VEGFA/defb29 tumor challenge and rechallenge. (FIG. 2A) Combination data from all survival experiments. Mice received treated ID8/VEGFA/defb29 cells, co-delivered IP with or without adjuvant. The panel shows the percent of mice (out of the total number that received that treatment in all experiments) that survived to day 100 to be rechallenged. (FIG. 2B) The panel shows the percent of mice that survived 60 days after being rechallenged. All groups with any mice surviving to rechallenge are included, as are all groups that included irradiated cells and all vehicle-treated mice. (FIG. 2A, FIG. 2B) Vehicle-treated n=24, irradiated cells n=20, irradiated cells+CPMV n=20, irradiated cells+MPLA n=8, irradiated cells+DMXAA n=8, freeze-thaw+CPMV n=4, freeze-thaw+MPLA n=3, HOCl+CPMV n=4.

FIG. 3: The survival benefit provided by the co-delivery of CPMV and irradiated cells is T cell-dependent. Irradiated ID8/VEGFA/defb29 cells were co-delivered IP with 100 μg CPMV to C57BL/6J (WT) or NU/J (nude) mice. Following two vaccinations seven days apart, mice were challenged with 5×106 live tumor cells. n=4 in all groups. Compared to WT vehicle: WT irradiated+CPMV p=0.007, nude irradiated+CPMV p=0.29, and nude vehicle p=0.10. Compared to nude vehicle, nude irradiated+CPMV p=0.009. p values compare survival curves with a log-rank (Mantel-Cox) test. All p values are compared to vehicle-treated controls unless otherwise noted. ** 0.001<p<0.01; ns p>0.05.

FIGS. 4A-4C: MPLA is an ineffective adjuvant against the ID8/VEGFA/defb29 murine ovarian cancer model. (FIG. 4A) Irradiated ID8/VEGFA/defb29 cells were codelivered IP with PBS or various doses of MPLA. n=5 for all groups; (FIG. 4B) Irradiated ID8/VEGFA/defb29 cells were codelivered with PBS or 100 μg MPLA. Antigen and adjuvant were codelivered either intraperitoneally (IP), intramuscularly (IM), or subcutaneously (SC). The vaccines in the vehicle and irradiated cell groups were delivered IP. n=5 for all groups; (FIG. 4C) Irradiated ID8/VEGFA/defb29 cells were codelivered IP with 100 μg MPLA. n=5 for all groups; (FIGS. 4A-4C) All mice received two vaccinations seven days apart. Mice were then challenged with live ID8/VEGFA/defb29 cells delivered IP. Once twice the average length of the survival of vehicle-treated control mice had passed, mice were rechallenged with an equivalent number of live ID8/VEGFA/defb29 cells. No results reached statistical significance (p<0.05).

### DETAILED DESCRIPTION

### Definitions

As it would be understood, the section or subsection headings as used herein is for organizational purposes only and are not to be construed as limiting or separating or both limiting and separating the subject matter described.

Throughout this disclosure, various publications, patents and published patent specifications are referenced by an identifying citation. The disclosures of these publications, patents and published patent specifications are hereby incorporated by reference into the present disclosure in their entireties to more fully describe the state of the art to which this invention pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human.

A “composition” as used herein, refers to an active agent, such as a compound as disclosed herein and a carrier, inert or active. The carrier can be, without limitation, solid such as a bead or resin, or liquid, such as phosphate buffered saline.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

Administration or treatment in “combination” refers to administering two agents such that their pharmacological effects are manifest at the same time. Combination does not require administration at the same time or substantially the same time, although combination can include such administrations.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor, or delay, slowing, or prevent of relapse. In one aspect, treatment excludes prophylaxis. In one aspect, treatment provides a longer progression free survival or a longer overall survival.

In one embodiment, the term “disease” or “disorder” as used herein refers to a cancer or a tumor (which are used interchangeably herein), a status of being diagnosed with such disease, a status of being suspect of having such disease, or a status of at high risk of having such disease. In one aspect, the cancer is ovarian cancer such as ovarian serous carcinoma.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. In some embodiments, the term “cancer” is used interchangeably with the term “tumor”. In one aspect, the cancer is ovarian cancer such as ovarian serous carcinoma.

As used herein, an ablative therapy is a treatment destroying or ablating cancer tumors. In one embodiment, the ablative therapy does not require invasive surgery. In other embodiments, the ablative therapy refers to removal of a tumor via surgery. In some embodiments, the step ablating the cancer includes immunotherapy of the cancer. Cancer immunotherapy is based on therapeutic interventions that aim to utilize the immune system to combat malignant diseases. It can be divided into unspecific approaches and specific approaches. Unspecific cancer immunotherapy aims at activating parts of the immune system generally, such as treatment with specific cytokines known to be effective in cancer immunotherapy (e.g. IL-2, interferon's, cytokine inducers).

The terms “oligonucleotide” or “polynucleotide” or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

Cowpea mosaic virus (CPMV) is a plant-infecting member of the order Picornavirales, with a relatively simple, non-enveloped capsid that has been extensively studied and a positive-sense, single-stranded RNA genome. For CPMV, the genome is bipartite, with RNA-1 (6 kb) and RNA-2 (3.5 kb) being separately encapsidated. CPMV has an icosahedral capsid structure, which is ˜30 nm in diameter and is formed from 60 copies each of a Large (L) and Small (S) coat protein. These two coat proteins are processed from a single RNA-2-encoded precursor polyprotein (VP60) by the action of the 24 K viral proteinase which is encoded by RNA-1. Thus capsid assembly, as well as viral infection, is dependent on the presence of both genomic segments in an infected plant cell.

The terms “CPMV” “CPMV virus” or “CPMV particles” are used interchangeably, referring to a CPMV comprising, or alternatively consisting essentially of, or yet consisting of a capsid and an RNA genome (which is also referred to herein as a viral genome) encapsidated in the capsid. In some embodiments, the CPMV particles have been treated, prepared and/or inactivated by a method as disclosed herein. In some embodiments, the CPMV particle further comprises a heterologous RNA, which is heterologous to (i.e., not naturally presented in) a native CPMV free of any human intervention.

In some embodiments, the CPMV can be substitute by another plant virus, for example another plant retrovirus. However, a bacteriophage or mammalian virus can be used in some embodiments of the invention. When a plant virus is used, in some embodiments the plant virus is a plant picornavirus. A plant picornavirus is a virus belonging to the family Secoaviridae, which together with mammalian picomaviruses belong to the order of the Picornavirales. Plant picomaviruses are relatively small, nonenveloped, positive-stranded RNA viruses with an icosahedral capsid. Plant picomaviruses have a number of additional properties that distinguish them from other picomaviruses, and are categorized as the subfamily secoviridae. In some embodiments, the virus particles are selected from the Comovirinae virus subfamily. Examples of viruses from the Comovirinae subfamily include Cowpea mosaic virus, Broad bean wilt virus 1, and Tobacco ringspot virus. In a further embodiment, the virus particles are from the Genus comovirus. A preferred example of a comovirus is the cowpea mosaic virus particles. Other suitable plant virus includes, but is not limited to bean pod mottle virus (BPMV) or rice tungro spherical virus.

The virus can be obtained according to various methods known to those skilled in the art. In embodiments where plant virus particles are used, the virus particles can be obtained from the extract of a plant infected by the plant virus or using the method disclosed herein. For example, cowpea mosaic virus can be grown in black eyed pea plants, which can be infected within 10 days of sowing seeds. Plants can be infected by, for example, coating the leaves with a liquid containing the virus, and then rubbing the leaves, preferably in the presence of an abrasive powder which wounds the leaf surface to allow penetration of the leaf and infection of the plant. Within a week or two after infection, leaves are harvested and viral nanoparticles are extracted. In the case of cowpea mosaic virus, 100 mg of virus can be obtained from as few as 50 plants. Procedures for obtaining plant picornavirus particles using extraction of an infected plant are known to those skilled in the art. See Wellink J., Meth Mol Biol, 8, 205-209 (1998). Procedures are also available for obtaining virus-like particles. Saunders et al., Virology, 393(2):329-37 (2009). The disclosures of both of these references are incorporated herein by reference.

In some embodiments, the CPMV or CMPV particle has a diameter of from about 15 nm to about 60 nm, or from about 15 nm to about 50 nm, or from about 10 nm to about 40 nm, or from about 20 or 25 nm to about 50 nm, or from about 20 or 25 nm to about 40 nm, or from about 15 nm to about 35 nm, or from about 15 nm to about 40 nm, or from about 15 nm to about 45 nm, or alternatively about 15 nm, or about 20 nm, or about 25 nm, or about 30 nm, or about 35 nm, or about 40 nm, or about 45 nm, or about 50 nm, or about 55 nm, or about 60 nm.

Modes for Carrying Out the Disclosure

Applicant used CPMV as an adjuvant to model treatment during cancer patients' periods of remission. Without being bound by theory, Applicant proposed using the tumor as a remission stage vaccine antigen source. By using the tumor as the antigen source, the vaccine is fully personalized. This approach is particularly attractive in the ovarian cancer context because the vast majority of serous ovarian cancer patients undergo surgical debulking, which generates hundreds of grams or multiple kilograms of patient tumor, which is currently discarded. Rather than discarding the tumor, it can be retained, disaggregated, inactivated to ensure cells cannot divide, and frozen for future use as the antigen source in a remission-stage vaccine. The inactivated tumor tissue will contain many of the tumor-associated antigens or neoantigens that would be carried by tumors during disease relapse. When combined with adjuvant, the treated tumor tissue could be administered to patients with the goal of preventing fatal relapse.

### Therapeutic Methods and Kits

In one aspect, provided is a method for inhibiting, delaying, slowing down, or preventing relapse of a cancer in a subject in need thereof. The method comprises, consists essentially of, or consist of administering to the subject: (a) an adjuvant which comprises, or consists essentially of, or consists of a cowpea mosaic virus (CPMV) particle, for example, an effective amount of the adjuvant; and (b) one or more cancer antigen(s) or a cancer cell comprising the one or more cancer antigen(s), for example, an effective amount of the antigen(s) or cancer cell, thereby delaying, slowing down, or preventing the relapse of the cancer in the subject. In one aspect, the cancer is a carcinoma or a sarcoma. In one aspect the cancer is a carcinoma. In one aspect, the cancer antigen(s) and or cancer cells are of the same type of cancer that the subject is suffering from, e.g., ovarian cancer antigens for the treatment of ovarian cancer. In one aspect, the cancer is ovarian cancer such as ovarian serous carcinoma. In one aspect, the antigens comprise, or consist essentially of, or yet further consist of cancer antigen(s) and or cancer cells. In one aspect the antigen(s) comprise, or alternatively consist essentially of, or yet further consist of carcinoma antigen(s) or sarcoma antigen(s). In one embodiment the antigen(s) comprise, or consist essentially of or yet further consist of ovarian cancer antigens for the treatment of ovarian cancer. In one aspect, the antigen(s) comprise, or consist essentially of, or yet further consist of ovarian serous carcinoma antigen(s). In one aspect, the cowpea mosaic virus (CPMV) particle is the only active anticancer agent in the adjuvant. In some embodiments, the CPMV or CMPV particle has a diameter of from about 15 nm to about 60 nm, or from about 15 nm to about 50 nm, or from about 10 nm to about 40 nm, or from about 20 or 25 nm to about 50 nm, or from about 20 or 25 nm to about 40 nm, or from about 15 nm to about 35 nm, or from about 15 nm to about 40 nm, or from about 15 nm to about 45 nm, or alternatively about 15 nm, or about 20 nm, or about 25 nm, or about 30 nm, or about 35 nm, or about 40 nm, or about 45 nm, or about 50 nm, or about 55 nm, or about 60 nm.

In another aspect, provided is a method for treating a cancer in a subject in need thereof. The method comprises, consists essentially of, or consist of administering to the subject: (a) an adjuvant which comprises, or consists essentially of, or consists of a cowpea mosaic virus (CPMV) particle, for example, an effective amount of the adjuvant; and (b) one or more cancer antigen(s) or a cancer cell comprising the one or more cancer antigen(s), for example, an effective amount of the antigen(s) or cancer cell, thereby treating the cancer in the subject. In one aspect, the cancer is a carcinoma or a sarcoma. In one aspect the cancer is a carcinoma. In one aspect, the antigens comprise, or consist essentially of, or yet further consist of cancer antigen(s) and or cancer cells. In one aspect the antigen(s) comprise, or alternatively consist essentially of, or yet further consist of carcinoma antigen(s) or sarcoma antigen(s). In one embodiment the antigen(s) comprise, or consist essentially of or yet further consist of ovarian cancer antigens for the treatment of ovarian cancer. In one aspect, the antigen(s) comprise, or consist essentially of, or yet further consist of ovarian serous carcinoma antigen(s). In one aspect, the cancer antigen(s) and or cancer cells are of the same type of cancer that the subject is suffering from, e.g., ovarian cancer antigens for the treatment of ovarian cancer. In one aspect, the cancer is ovarian cancer such as ovarian serous carcinoma. In one aspect, the cowpea mosaic virus (CPMV) particle is the only active anticancer agent in the adjuvant. In some embodiments, the CPMV or CPMV particle has a diameter of from about 15 nm to about 60 nm, or from about 15 nm to about 50 nm, or from about 10 nm to about 40 nm, or from about 20 or 25 nm to about 50 nm, or from about 20 or 25 nm to about 40 nm, or from about 15 nm to about 35 nm, or from about 15 nm to about 40 nm, or from about 15 nm to about 45 nm, or alternatively about 15 nm, or about 20 nm, or about 25 nm, or about 30 nm, or about 35 nm, or about 40 nm, or about 45 nm, or about 50 nm, or about 55 nm, or about 60 nm.

In yet another aspect, provided is a method for triggering or enhancing one or more of the following in a subject in need thereof: an immune response, innate immune responses, a T cell-dependent immune response, a T cell priming, a CD8+ T cell-dependent immune response, or an immunological memory. The method comprises, consists essentially of, or consist of administering to the subject (a) an adjuvant which comprises, or consists essentially of, or consists of a cowpea mosaic virus (CPMV) particle, for example, an effective amount of the adjuvant; and (b) one or more antigen(s), or a cell comprising the one or more antigen(s), for example, an effective amount of the antigen(s) or cancer cell, thereby triggering or enhancing one or more of the following in the subject: an immune response, a T cell-dependent immune response, a T cell priming, a CD8+ T cell-dependent immune response, or an immunological memory. In some embodiments, the immune response, T cell priming, or immunological memory is anti-cancer. Additionally or alternatively, the antigen(s) is a cancer antigen(s). In further embodiments, the cell comprising the one or more antigen(s) is a cancer cell. In one aspect, the cancer is a carcinoma or a sarcoma. In one aspect the cancer is a carcinoma. In one aspect, the cancer antigen(s) and or cancer cells are of the same type of cancer that the subject is suffering from, e.g., ovarian cancer antigens for the treatment of ovarian cancer. In one aspect, the cancer is ovarian cancer such as ovarian serous carcinoma. In one aspect, the antigens comprise, or consist essentially of, or yet further consist of cancer antigen(s) and or cancer cells. In one aspect the antigen(s) comprise, or alternatively consist essentially of, or yet further consist of carcinoma antigen(s) or sarcoma antigen(s). In one embodiment the antigen(s) comprise, or consist essentially of or yet further consist of ovarian cancer antigens for the treatment of ovarian cancer. In one aspect, the antigen(s) comprise, or consist essentially of, or yet further consist of ovarian serous carcinoma antigen(s). The vaccine is useful for the treatment of cancer such as ovarian cancer or for use in a method as disclosed herein. In one aspect, the cowpea mosaic virus (CPMV) particle is the only active anticancer agent in the adjuvant. In some embodiments, the CPMV or CPMV particle has a diameter of from about 15 nm to about 60 nm, or from about 15 nm to about 50 nm, or from about 10 nm to about 40 nm, or from about 20 or 25 nm to about 50 nm, or from about 20 or 25 nm to about 40 nm, or from about 15 nm to about 35 nm, or from about 15 nm to about 40 nm, or from about 15 nm to about 45 nm, or alternatively about 15 nm, or about 20 nm, or about 25 nm, or about 30 nm, or about 35 nm, or about 40 nm, or about 45 nm, or about 50 nm, or about 55 nm, or about 60 nm.

In some embodiments relating to any method(s), any kit(s), any composition(s), any aspect(s), or any other embodiment(s) as disclosed herein, the cell comprising the one or more antigen(s) is irradiated.

In some embodiments relating to any method(s), any kit(s), any composition(s), any aspect(s), or any other embodiment(s) as disclosed herein, the CPMV particle is non-replicating and noninfectious in the subject.

In some embodiments relating to any method(s), any kit(s), any composition(s), any aspect(s), or any other embodiment(s) as disclosed herein, the cancer is an ovarian cancer.

In some embodiments relating to any method(s), any kit(s), any composition(s), any aspect(s), or any other embodiment(s) as disclosed herein, the cancer cell is autologous or allogeneic to the subject. In some embodiments, the cancer cell is isolated from the subject or a tumor tissue resected from the subject.

In some embodiments relating to any method(s), any kit(s), any composition(s), any aspect(s), or any other embodiment(s) as disclosed herein, the cell comprising the one or more antigen(s) is not capable of dividing. In some embodiments, the cell is treated by one or more of: an irradiation, one or more of freeze and thaw cycles, a heat-shock, or a hypochlorous acid (HOCl) oxidization.

In some embodiments, a method as disclosed herein further comprises treating the cell by one or more of: an irradiation, one or more of freeze and thaw cycles, a heat-shock, or a hypochlorous acid (HOCl) oxidization.

In some embodiments, a method as disclosed herein further comprises isolating a cancer cell from the subject prior to the administration of (a) and (b). In further embodiments, a method as disclosed herein further comprises irradiating the cancer cell.

In some embodiments relating to any method(s), any kit(s), any composition(s), any aspect(s), or any other embodiment(s) as disclosed herein, the cell is irradiated with about 10 Gray to about 1000 Gray, or any other range(s) or value(s) falling within the range of about 10 Gray to about 1000 Gray, for example, about 100 Gray or about 70 Gray, prior to the administration.

In some embodiments relating to any method(s), any kit(s), any composition(s), any aspect(s), or any other embodiment(s) as disclosed herein, (a) and (b) as disclosed herein are administrated in one dose or in two separate doses. In some embodiments, (a) and (b) are administrated concurrently or sequentially. In some embodiments, the administration(s) of (a) and (b) is repeated for at least once, or at least twice, or more. In further embodiments, any two administrations are about 1 day to about 1 year apart, or about 1 week apart, or about 2 weeks apart, or about 3 weeks apart, or about 4 weeks apart, or about 1 month apart, or about 2 months apart, or about 3 months apart, or about 6 months apart.

In some embodiments relating to any method(s), any kit(s), any composition(s), any aspect(s), or any other embodiment(s) as disclosed herein, the subject is a cancer patient in a remission stage. Additionally or alternatively, the cancer in the subject is clinically undetectable.

In some embodiments relating to any method(s), any kit(s), any composition(s), any aspect(s), or any other embodiment(s) as disclosed herein, the subject is a cancer patient who has been treated by one or more of: an ablative therapy, a chemotherapy, a radiation therapy, an immune checkpoint blockade therapy, or another anti-cancer therapy.

In some embodiments, a method as disclosed herein further comprises treating the subject with one or more of: an ablative therapy, a chemotherapy, a radiation therapy, an immune checkpoint blockade therapy, or another anti-cancer therapy.

In some embodiments relating to any method(s), any kit(s), any composition(s), any aspect(s), or any other embodiment(s) as disclosed herein, (a) and (b) as disclosed herein are administered as a first line therapy, a second line therapy, a third line therapy or a fourth line therapy.

In one aspect, provided is a kit for use in a method as disclosed herein. The method comprises, consists essentially of, or consists of (a) an adjuvant which comprises, or consists essentially of, or consists of a cowpea mosaic virus (CPMV) particle; and an optional instruction for use. In some embodiments, the kit further comprises (b) one or more antigen(s), or a cell comprising the one or more antigen(s).

### Compositions

In another aspect, provided is a vaccine composition comprising, consisting essentially of, or consisting of (a) an adjuvant which comprises, or consists essentially of, or consists of a cowpea mosaic virus (CPMV) particle; and (b) one or more antigen(s) or a cell comprising the one or more antigen(s). In one aspect, the antigens comprise, or consist essentially of, or yet further consist of cancer antigen(s) and or cancer cells. In one aspect the antigen(s) comprise, or alternatively consist essentially of, or yet further consist of carcinoma antigen(s) or sarcoma antigen(s). In one embodiment the antigen(s) comprise, or consist essentially of or yet further consist of ovarian cancer antigens for the treatment of ovarian cancer. In one aspect, the antigen(s) comprise, or consist essentially of, or yet further consist of ovarian serous carcinoma antigen(s). In one aspect, the cowpea mosaic virus (CPMV) particle is the only active anticancer agent in the adjuvant. The vaccine is useful for the treatment of cancer such as ovarian cancer or for use in a method as disclosed herein.

In some embodiments relating to any method(s), any kit(s), any composition(s), any aspect(s), or any other embodiment(s) as disclosed herein, the CPMV particle is non-replicating and noninfectious in a subject.

In some embodiments relating to any method(s), any kit(s), any composition(s), any aspect(s), or any other embodiment(s) as disclosed herein, the cell is a cancer cell.

In some embodiments relating to any method(s), any kit(s), any composition(s), any aspect(s), or any other embodiment(s) as disclosed herein, the cell is an ovarian cancer or an ovarian serous carcinoma.

In some embodiments relating to any method(s), any kit(s), any composition(s), any aspect(s), or any other embodiment(s) as disclosed herein, the cancer cell is isolated from a subject. In some embodiments, the cancer cell is isolated from a tumor tissue resected from the subject, e.g., an ovarian cancer subject.

In some embodiments relating to any method(s), any kit(s), any composition(s), any aspect(s), or any other embodiment(s) as disclosed herein, the cell is not capable of dividing.

In some embodiments relating to any method(s), any kit(s), any composition(s), any aspect(s), or any other embodiment(s) as disclosed herein, the cell is treated by one or more of: irradiation, one or more of freeze and thaw cycles, heat-shock, or hypochlorous acid (HOCl) oxidization. In some embodiments, the cell is an irradiated cancer cell. In further embodiments, the cell is irradiated with about 10 Gray to about 1000 Gray, or any other range(s) or value(s) falling within the range of about 10 Gray to about 1000 Gray, for example about 100 Gray or about 70 Gray.

In some embodiments, a composition as disclosed herein further comprises a carrier. In some embodiments, a composition as disclosed herein further comprises a pharmaceutically acceptable carrier.

### Pharmaceutical Compositions

In another aspect, provided herein is a composition comprising, consisting essentially of, or consisting of an irradiated cell and a CMPV particle, and at least one pharmaceutically acceptable excipient. In one aspect, the cell comprises, or consists essentially of, or yet further consists of an irradiated cancer cell. In one aspect the cell comprises, or alternatively consists essentially of, or yet further consists of a carcinoma or a sarcoma. In one embodiment the cell comprises, or consists essentially of or yet further consists of an ovarian cancer cell or ovarian serous carcinoma cells. The composition is useful for the treatment of cancer such as ovarian cancer or for use in a method as disclosed herein.

Compositions, including pharmaceutical compositions comprising, consisting essentially of, or consisting of a component or a combination as described herein, can be manufactured by means of conventional mixing, dissolving, granulating, dragee-making levigating, emulsifying, encapsulating, entrapping, or lyophilization processes. The component or combination can be formulated in conventional manner using one or more physiologically acceptable carriers, diluents, excipients, or auxiliaries which facilitate processing of the component or combination provided herein into preparations which can be used pharmaceutically.

The component or combination of the present disclosure can be administered by parenteral (e.g., intramuscular, intraperitoneal, intravenous, ICV, intracisternal injection or infusion, subcutaneous injection, or implant), oral, by inhalation spray nasal, vaginal, rectal, sublingual, urethral (e.g., urethral suppository) or topical routes of administration (e.g., gel, ointment, cream, aerosol, etc.) and can be formulated in suitable dosage unit formulations containing conventional non-toxic pharmaceutically acceptable carriers, adjuvants, excipients, and vehicles appropriate for each route of administration.

In one embodiment, this technology relates to a composition comprising a component or a combination as described herein and a carrier.

In another embodiment, this technology relates to a pharmaceutical composition comprising a component or a combination as described herein and a pharmaceutically acceptable carrier.

In another embodiment, this technology relates to a pharmaceutical composition comprising a therapeutically effective amount of a component or a combination as described herein and a pharmaceutically acceptable carrier.

The pharmaceutical compositions for the administration of a component or a combination as disclosed herein can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition, each component provided herein is included in an amount sufficient to produce the desired effect. For example, pharmaceutical compositions of the present technology may take a form suitable for virtually any mode of administration, including, for example, topical, ocular, oral, buccal, systemic, nasal, injection, infusion, transdermal, rectal, and vaginal, or a form suitable for administration by inhalation or insufflation.

For topical administration, the component or the combination can be formulated as solutions, gels, ointments, creams, suspensions, etc., as is well-known in the art.

Systemic formulations include those designed for administration by injection (e.g., subcutaneous, intravenous, infusion, intramuscular, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the component or the combination provided herein can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the combination of compounds provided herein in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques well known to the skilled artisan. The pharmaceutical compositions of the present technology may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

### Dosages and Dosing Regimens

The appropriate amount and dosing regimen of the component or the combination, when present to be administered to the subject according to any of the methods disclosed herein, may be determined by one of ordinary skill in the art.

In some embodiments, the component or the combination as disclosed herein, may be administered to a subject in need thereof, either alone or as part of a pharmaceutically acceptable formulation, once a week, once a day, twice a day, three times a day, or four times a day, or even more frequently.

Administration of the component or the combination as disclosed herein may be effected by any method that enables delivery of the component or the combination to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14 16, 20, 24 or more hours or lasting for 1-7 days or more. Infusions can be administered by drip, continuous infusion, infusion pump, metering pump, depot formulation, or any other suitable means.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

### Experimental Methods

In this investigation, murine tumor cells were prepared in four different ways (irradiation, freeze-thawed lysates, heat-shocked lysates, and HOCl-oxidized lysates) to enhance the immunogenicity of the vaccine's antigen. [16, 36-43]. CPMV was used as an adjuvant and compared to monophosphoryl lipid A (MPLA), a bacterial cell wall component and potent TLR 4 agonist that is an FDA-approved vaccine adjuvant [44, 45]. Applicant also compared CPMV to DMXAA, a murine STING agonist and antivascular agent [46]. Following the administration of two vaccines, the mice were challenged with live murine ovarian cancer cells, with survival used as a measure of vaccine efficacy.

Reported here are the results of investigating multiple tumor cell treatments and adjuvants to identify the optimal tumor vaccine. Applicant observed that mice treated with the combination of CPMV and irradiated cells generated a robust T cell-dependent response, which induced superior survival against live tumor cell challenge and subsequent rechallenge, indicating the formation of immune memory. Here, Applicant shows that the codelivery of CPMV nanoparticles and lethally irradiated ovarian cancer cells could form the basis of an effective remission-stage ovarian cancer vaccine.

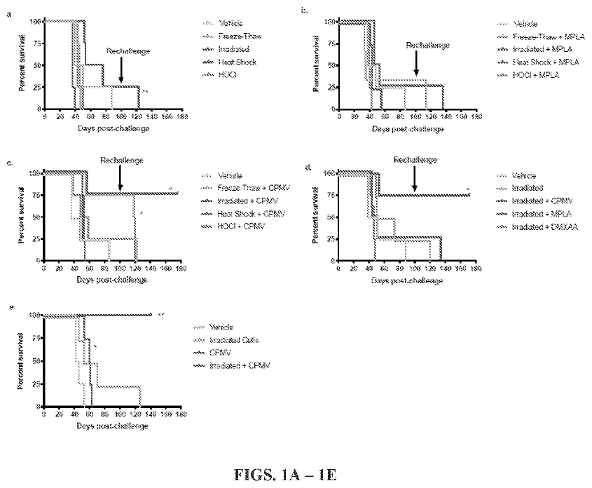

Without adjuvant, there was a modest survival advantage provided by the irradiated tumor cells, but none of the other cell preparations yielded a statistically significant survival benefit (FIG. 1A). This suggested that, of the preparations tested, radiation was the best option, and combination with adjuvant would strengthen its efficacy. MPLA is a weakly effective adjuvant against the ID8/VEGFA/defb29 murine ovarian cancer cell line when combined with irradiated cells (FIG. 1B). Indeed, none of the tumor antigen preparations in combination with MPLA conferred a significant survival advantage beyond the survival of mice given the same antigen preparations without adjuvant. MPLA was not an effective adjuvant in combination with irradiated tumor cells or freeze-thawed lysates, as it did not provide a survival benefit when compared to vehicle-treated mice (FIG. 1B) (p=0.59 and p=0.57, respectively). Mice treated with HOCl-oxidized cells and MPLA lived roughly as long as mice treated with HOCl-oxidized cells alone, showing that MPLA is not an effective adjuvant when combined with HOCl-oxidized cells (FIG. 1A, FIG. 1B) (p=0.82). Groups treated with heat-shocked lysates in combination with MPLA showed no significant difference between their survival and that of the vehicle-treated mice (p=0.81) (FIG. 1B). Because the vaccines that included MPLA as an adjuvant were ineffective, Applicant performed experiments changing the MPLA dose, the amount of antigen included in the vaccine, and the route of injection, but all formulations remained ineffective (FIG. 4). Applicant also investigated the combination of irradiated cells and DMXAA, a murine STING agonist, but it, too, did not extend mouse survival (p=0.28) (FIG. 1D).

Like MPLA, CPMV lacked consistent efficacy when combined with heat-shocked tumor lysates or HOCl-oxidized tumor lysates (FIG. 1C) (p=0.80 and p=0.53, respectively). The combination of CPMV and freeze-thawed lysates initially appeared effective, since 75% of the mice survived to rechallenge (FIG. 1C) (p=0.02). However, despite the significant survival benefit, the mice succumbed to their rechallenge within the expected forty-day timeline, suggesting that they did not mount a protective memory response (FIG. 1C). When combined with irradiated cells, CPMV significantly extended mouse survival and outperformed both MPLA and DMXAA as an adjuvant (FIG. 1C, FIG. 1D) (p=0.03 compared to MPLA and p=0.003 compared to DMXAA). Overall, then, compared to MPLA and DMXAA, CPMV provided a far superior survival benefit to mice in the ID8/VEGFA/defb29 model, particularly in combination with irradiated cells.

In the ID8/VEGFA/defb29 ovarian cancer model the best vaccine combined CPMV with irradiated tumor cells. Compared to the other vaccines tested, the co-administration of CPMV and irradiated cells extended survival the longest, was the only vaccine that enabled total tumor rejection in any mice, and provided mice with the ability to withstand rechallenge, justifying further characterization.

To confirm that both antigen and adjuvant, or in this case irradiated cells and CPMV, were necessary for vaccine efficacy, the survival of mice vaccinated with both irradiated cells and CPMV was compared to mice vaccinated with irradiated cells alone or CPMV alone. Both CPMV and irradiated cells were required to confer survival benefit against the ID8/VEGFA/defb29 cell line (FIG. 1E). At 140 days post-challenge, which is over three times as long as it generally takes for the vehicle-treated mice to reach the endpoint, all mice vaccinated with both irradiated cells and CPMV remained alive and tumor-free (p=0.006). In contrast, by day 65, all mice treated with CPMV alone had reached the endpoint criteria. While Applicant did observe that CPMV treatment alone provided a statistically significant survival benefit, the benefit was not comparable to that provided by the complete vaccine (FIG. 1E) (p=0.02). Similarly, all mice treated with irradiated cells alone succumbed to their cancer. Compared to CPMV alone and to irradiated cells alone, the combination vaccine significantly increased survival (p=0.007 for each), indicating that both irradiated cells and CPMV are necessary for full vaccine efficacy.

Applicant selected survival for 100 days after a challenge with ID8/VEGFA/defb29 tumor cells as the indication of rejection of the challenge. While vehicle-treated control mice reached endpoint criteria around 40 days post-challenge, over 40% of vaccine-treated mice in this study survived for 100 days. CPMV provided extremely dependable protection against rechallenge when combined with either irradiated cells or freeze-thawed cells, with 75-100% of mice given those vaccines surviving for 100 days or more with no signs of ascites development (FIGS. 1C-1E). However, other treatments also sporadically enabled the survival of challenged mice for 100 days at frequencies between 25 and 33% in some experiments, namely, irradiated cells only (FIG. 1A), freeze-thaw or irradiated+MPLA (FIGS. 1B, 1D), HOCl+CPMV (FIG. 1C), and irradiated cells only (FIG. 1A, FIG. 1D, FIG. 1E). In FIG. 2A, Applicant show the total percent survival of all mice at 100 days. These composite data clearly established the combination of CPMV and irradiated cells, or CPMV and freeze-thawed cells, as the best vaccine to mediate resistance to the primary challenge, and these treatments were roughly equal in protecting mice from primary tumor challenge.

While protection from the primary tumor challenge is an important assessment of vaccine efficacy, the establishment of protective immune memory is also very important. Accordingly, mice that survived the primary challenge for 100 days with no sign of ascites development were rechallenged to assess their ability to reject tumors months after vaccination, providing an indication of immune memory. The rechallenge data for each experiment and subsequent survival are shown in FIGS. 1A-1D. Applicant selected survival for at least 60 days following rechallenge as an indication of established, protective immune memory. Only mice treated with the combination of CPMV and irradiated cells survived 60 days following rechallenge (FIG. 1 and FIG. 2B). This study of longer-term protection clearly established the combination of CPMV and irradiated cells as superior to any other vaccine, including the combination of freeze-thawed cells and CPMV. Additionally, two mice vaccinated with CPMV and irradiated cells that survived 100 days after rechallenge (200 days after primary challenge) were rechallenged a second time at that 200 day mark, after which they survived to 300 days, when they were rechallenged for the third time. Both animals survived until 450 days after the primary challenge and had no ascites when the experiment was concluded (data not shown). Overall, these data show the unique ability of the vaccine combining CPMV and irradiated cells to establish long-term, protective memory against a mouse model of ovarian cancer.

### The Survival Benefit Provided by the Combination of CPMV and Irradiated Cells is T Cell-Dependent

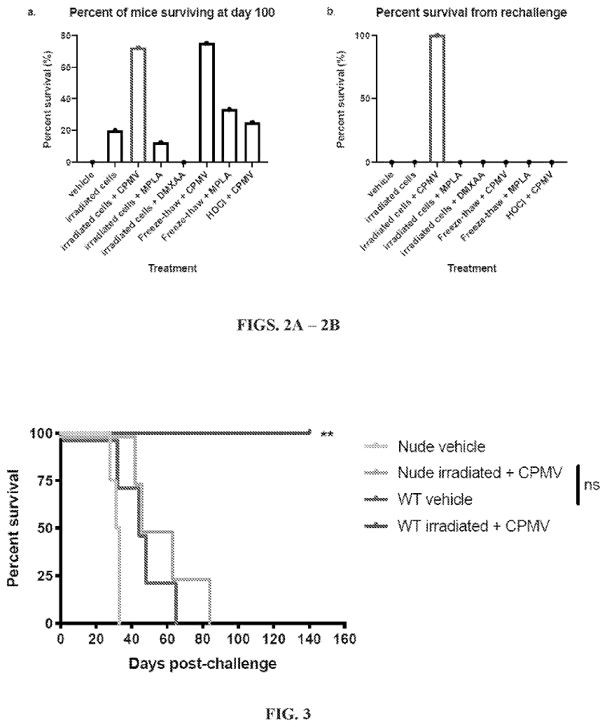

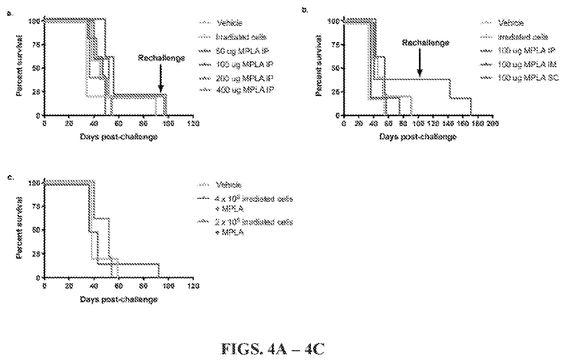

To begin to understand the immunological mechanisms of the vaccine combining irradiated cells and CPMV, the survival of vaccinated wild-type mice was compared to the survival of vaccinated nude mice that lack T cells. Comparing the vaccine's efficacy in nude mice to its efficacy in wild-type mice elucidates the importance of T cells to the anti-tumor immune response (FIG. 3).

The survival benefit conferred by the vaccine combining irradiated cells is T cell-dependent. Vehicle-treated nude mice succumbed to their tumors very quickly, likely because they lacked even the immune pressure of the anti-tumor T cells in unvaccinated mice (FIG. 3). However, the difference in survival between nude unvaccinated mice and wild-type unvaccinated mice was not significant (p=0.10). All vaccinated wild-type mice remained tumor-free at day 140, which was over three times the average survival of vehicle-treated mice, and they experienced a significant survival advantage compared to unvaccinated wild-type mice (p=0.007). The survival curves of the wild-type unvaccinated mice and the vaccinated nude mice closely mirrored one another, and there was no significant difference in the survival between these two groups (p=0.29). While all of the vaccinated wild-type mice rejected their tumors, none of the vaccinated nude mice rejected their tumors, providing clear evidence that the vaccine's immunological mechanism requires T cells. Applicant hypothesizes that the innate immune activation provided by CPMV allows a protective T cell response to be primed, ultimately causing tumor rejection.

### Materials and Methods

### Animals

Six-week-old female C57BL/6J and athymic nude (NU/J) mice were purchased from The Jackson Laboratory (Bar Harbor, ME, USA). All mice were housed in the Norris Cotton Cancer Center vivarium in accordance with Institutional Animal Care and Use Committee guidelines.

### Tumor Models

The ID8/VEGFA/defb29 murine ovarian serous carcinoma cell line was generated as previously described [49]. Cells were cultured at 37° C. in RPMI complete media (RPMI 1640 (HyClone, Marlborough, MA, USA) supplemented with 10% (v/v) fetal bovine serum (Gibco, Waltham, MA, USA), 1 mmol/L sodium pyruvate (Life Technologies, Waltham, MA, USA), 1% (v/v) penicillin/streptomycin mixture (Gibco, Waltham, MA, USA), and 2 mmol/L L-glutamine (Gibco, Waltham, MA, USA)). Cells were harvested and washed with RPMI 1640. Eight-week-old mice were challenged with 5×10⁶ tumor cells in 400 μL sterile PBS intraperitoneally on day 0 after receiving vaccines on days 14 and 7. After challenge, the mice were weighed regularly to monitor ascites formation. Mice were euthanized with carbon dioxide when they reached the humane endpoint of 33 g of weight, indicating significant ascites formation. Many surviving mice were rechallenged around 100 days after their initial tumor challenge. The mice were never given vaccines following tumor challenge or rechallenge.

### Vaccine Antigen Preparation

To prepare freeze-thawed lysates, the cells were washed with PBS and harvested. Cells were transferred to 15 mL conical tubes and resuspended in RPMI complete media. Tubes were submerged in a dry ice/ethanol slurry for 10 min. Cells were then allowed to thaw to room temperature in a room-temperature water bath. The freeze-thaw cycle was repeated five times. The freeze-thaw procedure was adapted from Chiang et al. (2011) and Herr et al. (2000) [43, 63]. Cell lysis was confirmed via trypan blue exclusion. Cells were resuspended in sterile PBS at a concentration of 25×10⁶ cells per mL. Lysates were injected intraperitoneally on days −14 and −7, with 5×10⁶ cells delivered to each mouse simultaneously with adjuvant injection.

To prepare irradiated cells, cells were washed with PBS and harvested. Cells were transferred to conical tubes in RPMI complete media and irradiated with 70 Gray (10 Gy per min for 7 min) ionizing gamma radiation from a cesium source. The preliminary experiments confirmed complete cell death following this radiation procedure. Cells were resuspended in sterile PBS at a concentration of 25×10⁶ cells per mL. Cells were placed on ice until just prior to vaccination. Irradiated cells were injected on days −14 and 7, with 5×10⁶ cells delivered to each mouse simultaneously with adjuvant injection.

To prepare heat-shocked lysates, cells were washed with PBS and harvested. Cells were resuspended in RPMI complete media and heat-shocked in a water bath at 43° C. for 30 min. Cells were removed from the water bath and incubated at 37° C. for 1 h. Cells were then subjected to five freeze-thaw cycles, as described above. The heat-shocked lysate procedure was adapted from Ito et al. (2005) [40]. Cells were resuspended in sterile PBS at a concentration of 25×10⁶ cells per mL. Lysates were injected intraperitoneally on days −14 and −7, with 5×10⁶ cells delivered to each mouse simultaneously with adjuvant injection.

To prepare hypochlorous acid-oxidized lysates, cells were washed with PBS and harvested. Cells were resuspended in 0.06 M HOCl in HBSS and incubated at 37° C. for 30 min. Cells were gently agitated to encourage oxidation, and then returned to the incubator for another 30 min. The HOCl-oxidation procedure was adapted from Chiang et al. (2006) [41]. Cells were centrifuged at 5000 rpm for 5 min and washed twice with PBS. Cells were subjected to five freeze-thaw cycles, as described above, before being resuspended in sterile PBS at a concentration of 25×10⁶ cells per mL. Lysates were injected intraperitoneally on days −14 and −7, with 5×10⁶ cells delivered to each mouse simultaneously with adjuvant injection.

Vaccines were entirely prophylactic; mice were never given vaccines after tumor challenge or rechallenge. Two doses were provided due to the preponderance of the literature suggesting that both primary and secondary immune responses are important for prophylactic vaccine efficacy [79, 80].

### Vaccine Adjuvant Preparation

MPLA (Sigma Aldrich, (St. Louis, MO, USA) was dissolved in 2.5% (v/v) sterile DMSO in ET-free PBS to a concentration of 0.50 μg MPLA/μL. Each mouse received 200 μL MPLA solution (100 μg MPLA) on day 14 and day 7, simultaneously with antigen injection. DMXAA was dissolved in 2.5% (v/v) sterile DMSO in ET-free PBS to a concentration of 1.25 μg MPLA/μL. Each mouse received 200 μL DMXAA solution (250 μg DMXAA) on days −14 and −7, simultaneously with antigen injection [81]. CPMV nanoparticles were prepared as previously described and were verified to have <50 endotoxin units per mg protein [82]. Briefly, as reported in [82], CPMV particles are obtained through infection of Vigna unguiculata plants followed by extraction and purification from the infected leaf tissue.

CPMV was diluted in PBS to a concentration of 100 μg per 200 μL PBS. Each mouse received 200 μL CPMV solution (100 μg CPMV) injected intraperitoneally on days −14 and −7, simultaneously with antigen injection.

### Statistical Analysis

All statistical analyses were performed with GraphPad Prism 8 (San Diego, CA, USA). All p values reported compare survival curves using the log-rank (Mantel-Cox) test. All experimental curves were compared to the relevant vehicle-treated controls unless otherwise stated.

### Results

CPMV is an Effective Adjuvant when Combined with Irradiated Syngeneic Ovarian Cancer Cells

MPLA is an FDA-approved vaccine adjuvant that stimulates TLR 4 on antigen-presenting cells (APCs) [44, 45]. Furthermore, because MPLA is widely used and easily obtained, it serves as a useful point of reference against which CPMV, the novel adjuvant, can be compared [47, 48]. Applicant compared the efficacies of these adjuvants in the ID8/VEGFA/defb29 ovarian cancer cell line, which is aggressive and closely mirrors human serous ovarian carcinoma; it is poorly immunogenic, metastasizes throughout the peritoneal cavity, and causes syngeneic C57BL/6J mice to rapidly develop acute ascites, making it an ideal model of serous ovarian cancer in humans [49].

To compare the efficacy of CPMV and MPLA as adjuvants against the ID8/VEGFA/defb29 cell line, they were co-administered with a variety of different antigen preparations. Each immune adjuvant was co-delivered intraperitoneally (IP) with irradiated tumor cells, freeze-thawed tumor lysates, heat-shocked tumor cell lysates, or HOCl-oxidized tumor cell lysates. All antigen preparations were selected because they dependably kill the tumor cells and have previously been found to increase tumor cell immunogenicity [16, 36-43]. Mice were given two identical vaccines one week apart, followed by a live tumor cell challenge one week after the second vaccine. Applicant followed the survival of mice given different antigen and adjuvant combinations after their live tumor cell challenge (FIG. 1).

### Discussion

Despite enormous advances in clinical cancer immunotherapies over the last two decades, none have shown clinical efficacy in treating ovarian carcinomas. Though a combination of surgical, chemotherapeutic, and radiological interventions often induces clinical remission, serous ovarian cancer generally returns. Accordingly, there is a significant need for patient-centered immunotherapies that could be delivered during patients' remission to prevent disease relapse [50]. Immunotherapies, including but not limited to vaccines, have the best opportunity to completely eliminate disease during remission, when low tumor burden leads to relatively weak tumor-mediated immunosuppression. As such, Applicant believe that a vaccine that actually cures disease, rather than one that extends survival, is best delivered during clinical remission before disease relapse. In order for that vaccine to induce a strong immune reaction, it must include an adjuvant. Furthermore, to specifically direct the vaccine against tumor cells, the vaccine must include an antigen source. If immunogenic tumor antigens overlap between the primary tumor removed during surgical debulking and the tumor present at relapse, then the primary tumor serves as a useful antigen source. Because a relatively large amount of tumor tissue is discarded from each patient's surgery, the tumor tissue removed during surgery serves as a patient-specific and readily available antigen source for a personalized cancer vaccine.

In this investigation, Applicant show that the combination of CPMV and irradiated murine ovarian cancer cells constitutes an effective, T cell-dependent prophylactic vaccination against an aggressive syngeneic mouse model of ovarian cancer. CPMV, an immunostimulatory plant viral nanoparticle that has previously shown promise as a therapeutic agent, was compared to MPLA, a TLR 4 agonist, and DMXAA, a murine STING agonist. CPMV was a consistently more effective adjuvant than either MPLA or DMXAA (FIG. 1). To determine the best antigen source for the vaccine, four different tumor preparations were compared-ionizing irradiation, freeze-thawed lysates, heat-shocked lysates, and hypochlorous acid-oxidized lysates—and Applicant observed that irradiated tumor cells were the most effective vaccine antigen. Together, the combination of CPMV and irradiated tumor cells enabled the majority of treated mice to reject the primary tumor challenge, as well as subsequent tumor challenges over a prolonged period.

Unsurprisingly, both CPMV (the adjuvant) and irradiated cells (the antigen) were necessary for vaccine efficacy, supporting the expected vaccine function (FIG. 1E). Most mice vaccinated with irradiated tumor cells and CPMV survived both the original tumor challenge and at least one rechallenge, with 70-75% of vaccinated mice surviving the initial challenge and all mice surviving rechallenge (FIG. 1 and FIG. 2). The remarkable efficacy of CPMV in extending survival in the highly aggressive ID8/VEGFA/defb29 model is consistent with other studies, which showed that CPMV moderately extended the survival of tumor-bearing mice when delivered therapeutically [32, 35, 51, 52]. Accordingly, the presents a novel application for CPMV as an effective adjuvant in remission-stage vaccines that block ovarian cancer relapse.

This sort of robust response is most often accomplished by CD8+ T cells, and this vaccine was rendered minimally effective in nude mice lacking T cells (FIG. 3). Furthermore, since vaccinated mice survived tumor rechallenges delivered 80-100 days after their original tumor challenge, they appear to have robust immunological memory against the ID8/VEGFA/defb29 tumor cell line (FIG. 2). While most tumor immunotherapies involve CD8+ T cells, the data Applicant present here are also consistent with a reliance upon CD4+ T cells, either for their own cytokine production or for their ability to help mount a protective B cell response; these possibilities cannot be ruled out [53]. Though more studies are necessary to thoroughly explain the vaccine's mechanisms, it is clear that the vaccine functions via a T cell-dependent immunological mechanism. These data corroborate earlier findings indicating that a vaccine consisting of CPMV conjugated to NY-ESO-1 induces an antigen-specific CD8+ T cell response [54].

Vaccines with whole tumor cell antigens have long been an area of interest in ovarian cancer immunotherapies [56-59]. Of the various antigen preparations that Applicant tested, the ionizing irradiation of tumor cells provided the best survival benefit (particularly in combination with CPMV) (FIG. 1A, FIG. 1C). Ionizing radiation has not been used extensively as an antigen preparation technique in the past. However, perhaps most famously, irradiated tumor cells expressing GM-CSF comprise GVAX, an early approach which helped galvanize the field of cancer immunotherapy [60]. Furthermore, irradiation has been found to increase dendritic cell activation and mouse survival compared to freeze-thawed lysates in a dendritic cell vaccine against mouse models of glioma and melanoma [36, 37]. Most probably, this is because gamma irradiation increases the expression of tumor antigens or oxidation-associated molecular patterns (specific types of danger-associated molecular patterns), which vigorously activate APCs [60, 61]. Furthermore, irradiation increases the expression of the T cell costimulatory molecule CD80 on a variety of tumor cells, which increases tumor cell immunogenicity [62]. These past findings are consistent with the reported results that irradiated tumor cells alone (without adjuvant) increased mouse survival (FIG. 1A).

Freeze-thawed lysates delivered in combination with CPMV initially conferred a survival benefit, but did not establish immunological memory, as mice succumbed to rechallenge (FIG. 1C). Freeze-thawed lysates are used regularly and are often used to prepare tumor cell lysates in successful dendritic cell vaccines [43, 59, 63]. For this reason, Applicant compared various different freeze-thawed lysates to irradiated cells. However, it is possible that freeze-thawed lysates can decrease the ability of dendritic cells to respond to TLR stimulation, which may explain why their combination with CPMV was not as effective as the combination of CPMV and irradiated cells was [64]. Other groups have found that treating ovarian cancer cells with HOCl has improved the ability of dendritic cells to prime anti-tumor T cell responses and extend survival [16, 41, 42, 65, 66]. These results did not corroborate the efficacy of HOCl oxidation as an antigen preparation technique. Because the heat treatment of cells causes an increased expression of immunogenic heat shock proteins, heat-shocked lysates have also been used to induce anti-tumor immune responses. Others have found that heat-shocking tumor cells can intensify the anti-tumor immune response, and they have made effective dendritic cell vaccines against colon cancer with heat-shocked tumor cells [38, 39]. Studies have even examined the optimal methods of heat-shocking tumor cells for vaccine preparation, which were used to inform our method of heat-shocking tumor cells [40]. The results from our study do not corroborate previous studies that have found heat-shocked lysates to be effective.

The results described here align with previous work regarding the role of T cells in ovarian cancer. Human patients with ovarian cancer are capable of mounting modest anti-tumor CD8+ T cell responses, though clinically these responses do not appear sufficient to protect patients; while it is very difficult to understand the level of response that exists early in disease development, the fact that clinical disease develops suggests that the T cell response is not sufficiently protective [67-71]. Perhaps the strategy modeled here, a combination of robust T cell priming and strategic delivery of the vaccine during times of low tumor burden, would invigorate T cell responses enough to prevent relapse in humans. Furthermore, there is reason to believe that genetically modifying the patient's own resected tumor tissue as a component of a personalized cancer vaccine would be useful in the clinical setting.

Mice that received two prophylactic vaccines consisting of irradiated cells and CPMV had a significant survival benefit. Applicant suggests here that, due to the low (clinically undetectable) tumor burden present during remission, prophylactic vaccine delivery provides a model for remission-stage vaccines. However, Applicant acknowledge that prophylactic vaccines do not perfectly model remission-stage vaccines; during clinical remission, the immune system is no longer naïve to cancer antigens, and tumor immune-editing can occur.

Mice treated with the best vaccine combination in this study, irradiated cells and CPMV, experienced a remarkable survival rate compared to other therapies reported in the literature with this model. In many of our trials, 70-75% of vaccinated mice survived the initial challenge and rechallenge, and our total combined cohort indicates that 100% of rechallenged mice survived their rechallenge (FIGS. 1-3). In contrast, other studies examining CPMV in this model cite a more modest survival benefit, with roughly 25% surviving challenge and rechallenge or, more frequently, no mice remaining tumor-free [33, 34, 51].

Immune checkpoint blockade therapies, which are widely used cancer immunotherapies, have at best a 15% overall response rate in ovarian cancer patients [3]. Some of the most promising murine ovarian cancer therapies to date combined various immunotherapies, such as a STING agonist with anti-PD-1 immune checkpoint blockade or GVAX, or FVAX, anti-41BB and anti-PD-1 or PD-L1 [73,74]. Compared to other murine vaccine studies, including dendritic cell vaccines, the vaccine developed in this study confers a much greater survival advantage [16, 24, 75]. In a study wherein mice were treated with a triple checkpoint blockade therapy, 20% of the mice remained tumor-free [76]. However, another group that engineered CAR T cells with the NKG2D receptor observed excellent mouse survival rates in ovarian cancer, and is moving toward clinical trials [77, 78]. One of the most promising prospective ovarian cancer vaccines targets Sp17 and utilizes CpG, a TLR 9 agonist, as an adjuvant [25]. While Applicant's vaccine provides comparable survival, the pre-dominance of tumor escape in single antigen vaccines has become apparent. Accordingly, the present investigation provides an important, effective direction for the development of multiple antigen-targeted ovarian cancer immunotherapies.

Thus, the results show that remission-stage ovarian cancer vaccines using irradiated tumor cells can effectively and significantly increase survival in a mouse model, suggesting the possibility of a similar potential in human serous ovarian cancer patients. These results suggest that, in other cancers in which patients frequently experience long periods of remission before their cancers recur, the development of inactivated tumor cell vaccines to be delivered during that period of remission could be useful. After testing a variety of tumor cell treatments and established as well as experimental adjuvants, Applicant found that only the combination of CPMV and irradiated cells enabled the vast majority of mice to respond to both the original tumor challenge and the rechallenge. Overall, the combination of irradiated cells and CPMV together provides a protective and T cell-dependent ovarian cancer vaccine against a mouse model of ovarian cancer, and opens doors for future studies in cancer immunotherapy.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entireties, to the same extent as if each were incorporated by reference individually. In case of conflict, the present specification, including definitions, will control.

Other aspects are set forth within the following claims.

### REFERENCES

Torre, L. A.; Trabert, B.; DeSantis, C. E.; Miller, K. D.; Samimi, G.; Runowicz, C. D.; Gaudet, M. M.; Jemal, A.; Siegel, R. L. Ovariancancer statistics, 2018. CA Cancer J. Clin. 2018, 68, 284-296.

Della Pepa, C.; Tonini, G.; Pisano, C.; Di Napoli, M.; Cecere, S. C.; Tambaro, R.; Facchini, G.; Pignata, S. Ovarian cancer standard of care: Are there real alternatives? Chin. J. Cancer 2015, 34, 17-27.

Hamanishi, J.; Mandai, M.; Ikeda, T.; Minami, M.; Kawaguchi, A.; Murayama, T.; Kanai, M.; Mori, Y.; Matsumoto, S.; Chikuma, S.; et al. Safety and Antitumor Activity of Anti-PD-1 Antibody, Nivolumab, in Patients With Platinum-Resistant Ovarian Cancer. J. Clin. Oncol. 2015, 33, 4015-4022.

Mahoney, K. M.; Rennert, P. D.; Freeman, G. J. Combination cancer immunotherapy and new immunomodulatory targets. Nat. Rev. Drug Discov. 2015, 14, 561-584.

Yang, Y. F.; Zou, J. P.; Mu, J.; Wijesuriya, R.; Ono, S.; Walunas, T.; Bluestone, J.; Fujiwara, H.; Hamaoka, T. Enhanced induction of antitumor T-cell responses by cytotoxic T lymphocyte-associated molecule-4 blockade: The effect is manifested only at the restricted tumor-bearing stages. Cancer Res. 1997, 57, 4036-4041.

Kudrin, A. Overview of cancer vaccines: Considerations for development. Hum. Vaccines Immunother. 2012, 8, 1335-1353.

Worzfeld, T.; Pogge von Strandmann, E.; Huber, M.; Adhikary, T.; Wagner, U.; Reinartz, S.; Muller, R. The Unique Molecular and Cellular Microenvironment of Ovarian Cancer. Front. Oncol. 2017, 7, 24.

Hagemann, T.; Wilson, J.; Burke, F.; Kulbe, H.; Li, N. F.; Pluddemann, A.; Charles, K.; Gordon, S.; Balkwill, F. R. Ovarian cancer cells polarize macrophages toward a tumor-associated phenotype. J. Immunol. 2006, 176, 5023-5032.

Landskron, J.; Helland, O.; Torgersen, K. M.; Aandahl, E. M.; Gjertsen, B. T.; Bjorge, L.; Tasken, K. Activated regulatory and memory T-cells accumulate in malignant ascites from ovarian carcinoma patients. Cancer Immunol. Immunother. 2015, 64, 337-347.

Chang, D. K.; Peterson, E.; Sun, J.; Goudie, C.; Drapkin, R. I.; Liu, J. F.; Matulonis, U.; Zhu, Q.; Marasco, W. A. Anti-CCR4 monoclonal antibody enhances antitumor immunity by modulating tumor-infiltrating Tregs in an ovarian cancer xenograft humanized mouse model. Oncoimmunology 2016, 5, e1090075.

Condamine, T.; Ramachandran, I.; Youn, J. I.; Gabrilovich, D. I. Regulation of tumor metastasis by myeloid-derived suppressor cells. Annu. Rev. Med. 2015, 66, 97-110.

Zhang, B.; Chen, F.; Xu, Q.; Han, L.; Xu, J.; Gao, L.; Sun, X.; Li, Y.; Li, Y.; Qian, M.; et al. Revisiting ovarian cancer microenvironment: A friend or a foe? Protein Cell 2018, 9, 674-692.

Daniel, D.; Urs, S.; Guley, K.; Krueger, S.; Draper, D.; Wong, A.; Evens, H.; Higginbottom, C.; Saims, D.; Wise, S.; et al. Abstract5691: Evaluation of immunomodulatory agents in classically immunologically ‘cold’ cancers using syngeneic mouse models of breast and ovarian cancer. Cancer Res. 2018, 78, 5691.

Hu, Z.; Ott, P. A.; Wu, C. J. Towards personalized, tumour-specific, therapeutic vaccines for cancer. Nat. Rev. Immunol. 2018, 18, 168-182.

Gray, H. J.; Benigno, B.; Berek, J.; Chang, J.; Mason, J.; Mileshkin, L.; Mitchell, P.; Moradi, M.; Recio, F. O.; Michener, C. M.; et al. Progression-free and overall survival in ovarian cancer patients treated with CVac, a mucin 1 dendritic cell therapy in a randomized phase 2 trial. J. Immunother. Cancer 2016, 4, 34.

Chiang, C. L.; Kandalaft, L. E.; Tanyi, J.; Hagemann, A. R.; Motz, G. T.; Svoronos, N.; Montone, K.; Mantia-Smaldone, G. M.; Smith, L.; Nisenbaum, H. L.; et al. A dendritic cell vaccine pulsed with autologous hypochlorous acid-oxidized ovarian cancer lysate primes effective broad antitumor immunity: From bench to bedside. Clin. Cancer Res. 2013, 19, 4801-4815.

Raza, A.; Merhi, M.; Inchakalody, V. P.; Krishnankutty, R.; Relecom, A.; Uddin, S.; Dermime, S. Unleashing the immune response to NY-ESO-1 cancer testis antigen as a potential target for cancer immunotherapy. J. Transl. Med. 2020, 18, 140.

Sabbatini, P.; Tsuji, T.; Ferran, L.; Ritter, E.; Sedrak, C.; Tuballes, K.; Jungbluth, A. A.; Ritter, G.; Aghajanian, C.; Bell-McGuinn, K.; et al. Phase I trial of overlapping long peptides from a tumor self-antigen and poly-ICLC shows rapid induction of integrated immune response in ovarian cancer patients. Clin. Cancer Res. 2012, 18, 6497-6508.

Ishihara, M.; Tono, Y.; Miyahara, Y.; Muraoka, D.; Harada, N.; Kageyama, S.; Sasaki, T.; Hori, Y.; Soga, N.; Uchida, K.; et al. First-in-human phase I clinical trial of the NY-ESO-1 protein cancer vaccine with NOD2 and TLR9 stimulants in patients with NY-ESO-1-expressing refractory solid tumors. Cancer Immunol. Immunother. 2020, 69, 663-675.

Xiang, S. D.; Gao, Q.; Wilson, K. L.; Heyerick, A.; Plebanski, M. A Nanoparticle Based Spl17 Peptide Vaccine Exposes New Immuno-Dominant and Species Cross-reactive B Cell Epitopes. Vaccines 2015, 3, 875-893.

Song, J. X.; Cao, W. L.; Li, F. Q.; Shi, L. N.; Jia, X. Anti-Sp17 monoclonal antibody with antibody-dependent cell-mediated cytotoxicity and complement-dependent cytotoxicity activities against human ovarian cancer cells. Med. Oncol. 2012, 29, 2923-2931.

Brunette, L. L.; Mhawech-Fauceglia, P. Y.; Ji, L.; Skeate, J. G.; Brand, H. E.; Lawrenson, K.; Walia, S.; Chiriva-Internati, M.; Groshen, S.; Roman, L. D.; et al. Validity and prognostic significance of sperm protein 17 as a tumor biomarker for epithelial ovarian cancer: A retrospective study. BMC Cancer 2018, 18, 970.

Gulley, J. L.; Arlen, P. M.; Tsang, K. Y.; Yokokawa, J.; Palena, C.; Poole, D. J.; Remondo, C.; Cereda, V.; Jones, J. L.; Pazdur, M. P.; et al. Pilot study of vaccination with recombinant CEA-MUC-1-TRICOM poxviral-based vaccines in patients with metastatic carcinoma. Clin. Cancer Res. 2008, 14, 3060-3069.

Wu, D.; Yu, X.; Wang, J.; Hui, X.; Zhang, Y.; Cai, Y.; Ren, M.; Guo, M.; Zhao, F.; Dou, J. Ovarian Cancer Stem Cells with High RORI Expression Serve as a New Prophylactic Vaccine for Ovarian Cancer. J. Immunol. Res. 2019, 2019, 9394615.

Chiriva-Internati, M.; Yu, Y.; Mirandola, L.; Jenkins, M. R.; Chapman, C.; Cannon, M.; Cobos, E.; Kast, W. M. Cancer testis antigen vaccination affords long-term protection in a murine model of ovarian cancer. PLoS ONE 2010, 5, e10471.

Yildiz, I.; Lee, K. L.; Chen, K.; Shukla, S.; Steinmetz, N. F. Infusion of imaging and therapeutic molecules into the plant virus-based carrier cowpea mosaic virus: Cargo-loading and delivery. J. Control. Release 2013, 172, 568-578.

Albakri, M. M.; Veliz, F. A.; Fiering, S. N.; Steinmetz, N. F.; Sieg, S. F. Endosomal toll-like receptors play a key role in activation of primary human monocytes by cowpea mosaic virus. Immunology 2020, 159, 183-192.

Lizotte, P. H.; Wen, A. M.; Sheen, M. R.; Fields, J.; Rojanasopondist, P.; Steinmetz, N. F.; Fiering, S. In situ vaccination with cowpea mosaic virus nanoparticles suppresses metastatic cancer. Nat. Nanotechnol. 2016, 11, 295-303.

Murray, A. A.; Wang, C.; Fiering, S.; Steinmetz, N. F. In Situ Vaccination with Cowpea vs Tobacco Mosaic Virus against Melanoma. Mol. Pharm. 2018, 15, 3700-3716.

Kerstetter-Fogle, A.; Shukla, S.; Wang, C.; Beiss, V.; Harris, P. L. R.; Sloan, A. E.; Steinmetz, N. F. Plant Virus-Like Particle In Situ Vaccine for Intracranial Glioma Immunotherapy. Cancers 2019, 11, 515.

Cai, H.; Wang, C.; Shukla, S.; Steinmetz, N. F. Cowpea Mosaic Virus Immunotherapy Combined with Cyclophosphamide Reduces Breast Cancer Tumor Burden and Inhibits Lung Metastasis. Adv. Sci. (Weinh.) 2019, 6, 1802281.

Czapar, A. E.; Tiu, B. D. B.; Veliz, F. A.; Pokorski, J. K.; Steinmetz, N. F. Slow-Release Formulation of Cowpea Mosaic Virus for In Situ Vaccine Delivery to Treat Ovarian Cancer. Adv. Sci. (Weinh.) 2018, 5, 1700991.

Shukla, S.; Wang, C.; Beiss, V.; Steinmetz, N. F. Antibody Response against Cowpea Mosaic Viral Nanoparticles Improves In Situ Vaccine Efficacy in Ovarian Cancer. ACS Nano 2020, 14, 2994-3003.

Wang, C.; Fiering, S. N.; Steinmetz, N. F. Cowpea Mosaic Virus Promotes Anti-Tumor Activity and Immune Memory in a Mouse Ovarian Tumor Model. Adv. Ther. 2019, 2.

Patel, R.; Czapar, A. E.; Fiering, S.; Oleinick, N. L.; Steinmetz, N. F. Radiation Therapy Combined with Cowpea Mosaic Virus Nanoparticle in Situ Vaccination Initiates Immune-Mediated Tumor Regression. ACS Omega 2018, 3, 3702-3707.

Vandenberk, L.; Garg, A. D.; Verschuere, T.; Koks, C.; Belmans, J.; Beullens, M.; Agostinis, P.; De Vleeschouwer, S.; Van Gool, S. W. Irradiation of necrotic cancer cells, employed for pulsing dendritic cells (DCs), potentiates DC vaccine-induced antitumor immunity against high-grade glioma. Oncoimmunology 2016, 5, e1083669.

Prasad, S. J.; Farrand, K. J.; Matthews, S. A.; Chang, J. H.; McHugh, R. S.; Ronchese, F. Dendritic cells loaded with stressed tumor cells elicit long-lasting protective tumor immunity in mice depleted of CD4+CD25+ regulatory T cells. J. Immunol. 2005, 174, 90-98.

Koido, S.; Hara, E.; Homma, S.; Mitsunaga, M.; Takahara, A.; Nagasaki, E.; Kawahara, H.; Watanabe, M.; Toyama, Y.; Yanagisawa, S.; et al. Synergistic induction of antigen-specific CTL by fusions of TLR-stimulated dendritic cells and heat-stressed tumor cells. J. Immunol. 2007, 179, 4874-4883.

Ying, M.; Zhen, Q.; Liu, S.; Gong, F.; Xie, Y. Treatment of established colon carcinoma-bearing mice by dendritic cells pulsed with lysates of heat-treated tumor cells. Sci. China C Life Sci. 2009, 52, 831-835.

Ito, A.; Fujioka, M.; Tanaka, K.; Kobayashi, T.; Honda, H. Screening of cytokines to enhance vaccine effects of heat shock protein70-rich tumor cell lysate. J. Biosci. Bioeng. 2005, 100, 36-42.

Chiang, C. L.; Ledermann, J. A.; Rad, A. N.; Katz, D. R.; Chain, B. M. Hypochlorous acid enhances immunogenicity and uptake of allogeneic ovarian tumor cells by dendritic cells to cross-prime tumor-specific T cells. Cancer Immunol. Immunother. 2006, 55, 1384-1395.

Chiang, C. L.; Ledermann, J. A.; Aitkens, E.; Benjamin, E.; Katz, D. R.; Chain, B. M. Oxidation of ovarian epithelial cancer cells by hypochlorous acid enhances immunogenicity and stimulates T cells that recognize autologous primary tumor. Clin. Cancer Res. 2008, 14, 4898-4907.

Chiang, C. L.; Hagemann, A. R.; Leskowitz, R.; Mick, R.; Garrabrant, T.; Czerniecki, B. J.; Kandalaft, L. E.; Powell, D. J., Jr.; Coukos, G. Day-4 myeloid dendritic cells pulsed with whole tumor lysate are highly immunogenic and elicit potent anti-tumor responses. PLoS ONE 2011, 6, e28732.

Cluff, C. W. Monophosphoryl lipid A (MPL) as an adjuvant for anti-cancer vaccines: Clinical results. Lipid A Cancer Ther. 2009, 667, 111-123.

Mata-Haro, V.; Cekic, C.; Martin, M.; Chilton, P. M.; Casella, C. R.; Mitchell, T. C. The vaccine adjuvant monophosphoryl lipid A as a TRIF-biased agonist of TLR4. Science 2007, 316, 1628-1632.

Weiss, J. M.; Guerin, M. V.; Regnier, F.; Renault, G.; Galy-Fauroux, I.; Vimeux, L.; Feuillet, V.; Peranzoni, E.; Thoreau, M.; Trautmann, A.; et al. The STING agonist DMXAA triggers a cooperation between T lymphocytes and myeloid cells that leads to tumor regression. Oncoimmunology 2017, 6, e1346765.

Shi, M.; Chen, X.; Ye, K.; Yao, Y.; Li, Y. Application potential of toll-like receptors in cancer immunotherapy: Systematic review. Medicine (Baltim.) 2016, 95, e3951.

Srivastava, A. K.; Dinc, G.; Sharma, R. K.; Yolcu, E. S.; Zhao, H.; Shirwan, H. SA-4-1BBL and monophosphoryl lipid A constitute an efficacious combination adjuvant for cancer vaccines. Cancer Res. 2014, 74, 6441-6451.

Conejo-Garcia, J. R.; Benencia, F.; Courreges, M. C.; Kang, E.; Mohamed-Hadley, A.; Buckanovich, R. J.; Holtz, D. O.; Jenkins, A.; Na, H.; Zhang, L.; et al. Tumor-infiltrating dendritic cell precursors recruited by a beta-defensin contribute to vasculogenesis under the influence of Vegf-A. Nat. Med. 2004, 10, 950-958.

Chu, C. S.; Boyer, J.; Schullery, D. S.; Gimotty, P. A.; Gamerman, V.; Bender, J.; Levine, B. L.; Coukos, G.; Rubin, S. C.; Morgan, M. A.; et al. Phase I/II randomized trial of dendritic cell vaccination with or without cyclophosphamide for consolidation therapy of advanced ovarian cancer in first or second remission. Cancer Immunol. Immunother. 2012, 61, 629-641.

Wang, C.; Beiss, V.; Steinmetz, N. F. Cowpea Mosaic Virus Nanoparticles and Empty Virus-Like Particles Show Distinct but Overlapping Immunostimulatory Properties. J. Virol. 2019, 93.

Shukla, S.; Wang, C.; Beiss, V.; Cai, H.; Washington, T., 2nd; Murray, A. A.; Gong, X.; Zhao, Z.; Masarapu, H.; Zlotnick, A.; et al. The unique potency of Cowpea mosaic virus (CPMV) in situ cancer vaccine. Biomater. Sci. 2020, 8, 5489-5503.

Nielsen, J. S.; Sahota, R. A.; Milne, K.; Kost, SE.; Nesslinger, N.J.; Watson, P. H.; Nelson, B. H. CD20+ tumor-infiltrating lymphocytes have an atypical CD27− memory phenotype and together with CD8+ T cells promote favorable prognosis in ovarian cancer. Clin. Cancer Res. 2012, 18, 3281-3292.

Patel, B. K.; Wang, C.; Lorens, B.; Levine, A. D.; Steinmetz, N. F.; Shukla, S. Cowpea Mosaic Virus (CPMV)-Based Cancer Testis Antigen NY-ESO-1 Vaccine Elicits an Antigen-Specific Cytotoxic T Cell Response. ACS Appl. Bio Mater. 2020, 3, 4179-4187.

Adams, S. F.; Grimm, A. J.; Chiang, C. L.; Mookerjee, A.; Flies, D.; Jean, S.; McCann, G. A.; Michaux, J.; Pak, H.; Huber, F.; et al. Rapid tumor vaccine using Toll-like receptor-activated ovarian cancer ascites monocytes. J. Immunother. Cancer 2020, 8.

Chiang, C. L.; Benencia, F.; Coukos, G. Whole tumor antigen vaccines. Semin. Immunol. 2010, 22, 132-143.

Chiang, C. L.; Coukos, G.; Kandalaft, L. E. Whole Tumor Antigen Vaccines: Where Are Applicant? Vaccines 2015, 3, 344-372.

Ophir, E.; Bobisse, S.; Coukos, G.; Harari, A.; Kandalaft, L. E. Personalized approaches to active immunotherapy in cancer. Biochim. Biophys. Acta 2016, 1865, 72-82.

Chiang, C. L.; Kandalaft, L. E.; Coukos, G. Adjuvants for enhancing the immunogenicity of whole tumor cell vaccines. Int. Rev. Immunol. 2011, 30, 150-182.

Dranoff, G.; Jaffee, E.; Lazenby, A.; Golumbek, P.; Levitsky, H.; Brose, K.; Jackson, V.; Hamada, H.; Pardoll, D.; Mulligan, R. C. Vaccination with irradiated tumor cells engineered to secrete murine granulocyte-macrophage colony-stimulating factor stimulates potent, specific, and long-lasting anti-tumor immunity. Proc. Natl. Acad. Sci. USA 1993, 90, 3539-3543.

Vandenberk, L.; Garg, A. D.; Agostinis, P.; Verschuere, T.; Koks, C.; De Vleeschouwer, S.; Van Gool, S. Irradiation of necrotic tumor cells used to pulse dendritic cells (DCs) potentiates DC vaccine-induced anti-tumor immunity in a mouse model of high-grade glioma. J. Immunother. Cancer 2014, 2.

Morel, A.; Fernandez, N.; De La Coste, A.; Haddada, H.; Viguier, M.; Polla, B. S.; Antoine, B.; Kahn, A. Gamma-ray irradiation induces B7.1 costimulatory molecule neoexpression in various murine tumor cells. Cancer Immunol. Immunother. 1998, 46, 277-282.

Herr, W.; Ranieri, E.; Olson, W.; Zarour, H.; Gesualdo, L.; Storkus, W. J. Mature dendritic cells pulsed with freeze-thaw cell lysates define an effective in vitro vaccine designed to elicit EBV-specific CD4(+) and CD8(+) T lymphocyte responses. Blood 2000, 96, 1857-1864.

Tirapu, I.; Lewis, A.; Kreutz, M.; McLinden, H.; Diebold, S. S. Freeze-and-thaw-disrupted tumour cells impair the responsiveness of DC to TLR stimulation. Eur. J. Immunol. 2008, 38, 2740-2750.

Kandalaft, L. E.; Powell, D. J., Jr.; Chiang, C. L.; Tanyi, J.; Kim, S.; Bosch, M.; Montone, K.; Mick, R.; Levine, B. L.; Torigian, D. A.; et al. Autologous lysate-pulsed dendritic cell vaccination followed by adoptive transfer of vaccine-primed ex vivo co-stimulated T cells in recurrent ovarian cancer. Oncoimmunology 2013, 2, e22664.

Tanyi, J. L.; Bobisse, S.; Ophir, E.; Tuyaerts, S.; Roberti, A.; Genolet, R.; Baumgartner, P.; Stevenson, B. J.; Iseli, C.; Dangaj, D.; et al. Personalized cancer vaccine effectively mobilizes antitumor T cell immunity in ovarian cancer. Sci. Transl. Med. 2018, 10.

Webb, J. R.; Milne, K.; Watson, P.; Deleeuw, R. J.; Nelson, B. H. Tumor-infiltrating lymphocytes expressing the tissue resident memory marker CD103 are associated with increased survival in high-grade serous ovarian cancer. Clin. Cancer Res. 2014, 20, 434-444.

Consortium, O. T. T. A.; Goode, E. L.; Block, M. S.; Kalli, K. R.; Vierkant, R. A.; Chen, W.; Fogarty, Z. C.; Gentry-Maharaj, A.; Toloczko, A.; Hein, A.; et al. Dose-Response Association of CD8+ Tumor-Infiltrating Lymphocytes and Survival Time in High-Grade Serous Ovarian Cancer. JAMA Oncol. 2017, 3, e173290.

Stanske, M.; Wienert, S.; Castillo-Tong, D. C.; Kreuzinger, C.; Vergote, I.; Lambrechts, S.; Gabra, H.; Gourley, C.; Ganapathi, R. N.; Kolaschinski, I.; et al. Dynamics of the Intratumoral Immune Response during Progression of High-Grade Serous Ovarian Cancer. Neoplasia 2018, 20, 280-288.

Wick, D. A.; Webb, J. R.; Nielsen, J. S.; Martin, S. D.; Kroeger, D. R.; Milne, K.; Castellarin, M.; Twumasi-Boateng, K.; Watson, P. H.; Holt, R. A.; et al. Surveillance of the tumor mutanome by T cells during progression from primary to recurrent ovarian cancer. Clin. Cancer Res. 2014, 20, 1125-1134.

Deniger, D. C.; Pasetto, A.; Prickett, T. D.; Gartner, J. J.; Bharathan, M.; Tran, E.; Robbins, P. F.; Rosenberg, S. A. Mutated Tumor Neoantigens Are Recognized by Tumor Infiltrating Lymphocytes from Metastatic Ovarian Cancer. Cancer Immunother. Cancer Vaccines II 2016, 24, S155.

Oh, J.; Barve, M.; Matthews, C. M.; Koon, E. C.; Heffernan, T. P.; Fine, B.; Grosen, E.; Bergman, M. K.; Fleming, E. L.; DeMars, L. R.; et al. Phase II study of Vigil(R) DNA engineered immunotherapy as maintenance in advanced stage ovarian cancer. Gynecol. Oncol. 2016, 143, 504-510.

Ghaffari, A.; Peterson, N.; Khalaj, K.; Vitkin, N.; Robinson, A.; Francis, J. A.; Koti, M. STING agonist therapy in combination with PD-1 immune checkpoint blockade enhances response to carboplatin chemotherapy in high-grade serous ovarian cancer. Br. J. Cancer 2018, 119, 440-449.

Duraiswamy, J.; Freeman, G. J.; Coukos, G. Therapeutic PD-1 pathway blockade augments with other modalities of immunother-apy T-cell function to prevent immune decline in ovarian cancer. Cancer Res. 2013, 73, 6900-6912.

Yuan, J.; Kashiwagi, S.; Reeves, P.; Nezivar, J.; Yang, Y.; Arrifin, N. H.; Nguyen, M.; Jean-Mary, G.; Tong, X.; Uppal, P.; et al. A novel mycobacterial Hsp70-containing fusion protein targeting mesothelin augments antitumor immunity and prolongs survival in murine models of ovarian cancer and mesothelioma. J. Hematol. Oncol. 2014, 7, 15.

Huang, R. Y.; Francois, A.; McGray, A. R.; Miliotto, A.; Odunsi, K. Compensatory upregulation of PD-1, LAG-3, and CTLA-4 limits the efficacy of single-agent checkpoint blockade in metastatic ovarian cancer. Oncoimmunology 2017, 6, e1249561.

Barber, A.; Zhang, T.; Sentman, C. L. Immunotherapy with chimeric NKG2D receptors leads to long-term tumor-free survival and development of host antitumor immunity in murine ovarian cancer. J. Immunol. 2008, 180, 72-78.

Spear, P.; Barber, A.; Sentman, C. L. Collaboration of chimeric antigen receptor (CAR)-expressing T cells and host T cells for optimal elimination of established ovarian tumors. Oncoimmunology 2013, 2, e23564.

Clem, A. S. Fundamentals of vaccine immunology. J. Glob. Infect. Dis. 2011, 3, 73-78.

Watson, B.; Boardman, C.; Laufer, D.; Piercy, S.; Tustin, N.; Olaleye, D.; Cnaan, A.; Starr, S. E. Humoral and cell-mediated immune responses in healthy children after one or two doses of varicella vaccine. Clin. Infect. Dis. 1995, 20, 316-319.

Jing, W.; McAllister, D.; Vonderhaar, E. P.; Palen, K.; Riese, M. J.; Gershan, J.; Johnson, B. D.; Dwinell, M. B. STING agonist inflames the pancreatic cancer immune microenvironment and reduces tumor burden in mouse models. J. Immunother. Cancer 2019, 7, 115.

Murray, A. A.; Sheen, M. R.; Veliz, F. A.; Fiering, S. N.; Steinmetz, N. F. In Situ Vaccination of Tumors Using Plant Viral Nanoparticles. Methods Mol. Biol. 2019, 2000, 111-124.

1. A method for inhibiting, delaying, slowing down, or preventing relapse of cancer in a subject in need thereof, comprising administering to the subject: (a) an adjuvant which comprises a cowpea mosaic virus (CPMV) particle; and (b) one or more cancer antigen(s) or a cancer cell comprising the one or more cancer antigen(s), thereby delaying, slowing down, or preventing the relapse of the cancer in the subject.

2. A method for treating cancer in a subject in need thereof, comprising administering to the subject: (a) an adjuvant which comprises a cowpea mosaic virus (CPMV) particle; and (b) one or more cancer antigen(s) or a cancer cell comprising the one or more cancer antigen(s), thereby treating the cancer in the subject.

3. A method for triggering or enhancing one or more of the following in a subject in need thereof: an immune response, innate immune responses, a T cell-dependent immune response, a T cell priming, a CD8+ T cell-dependent immune response, or an immunological memory, comprising administering to the subject (a) an adjuvant which comprises a cowpea mosaic virus (CPMV) particle; and (b) one or more antigen(s), or a cell comprising the one or more antigen(s), thereby triggering or enhancing one or more of the following in the subject: an immune response, a T cell-dependent immune response, a T cell priming, a CD8+ T cell-dependent immune response, or an immunological memory.

4. The method of claim 1, wherein the cell comprising the one or more antigen(s) is irradiated.

5. The method of claim 1, wherein the CPMV particle is non-replicating and noninfectious in the subject.

6. The method of claim 1, wherein the subject is in remission.

7. The method of claim 1, wherein the cancer cell is autologous or allogeneic to the subject.

8. The method of claim 7, wherein the cancer is a carcinoma or a sarcoma and the cancer cell is isolated from the subject or a tumor tissue resected from the subject.

9. The method of claim 1, wherein the cancer cell or antigen(s) comprise ovarian cancer cells or ovarian cancer antigen(s) and the subject is an ovarian cancer patient.

10. The method of claim 1, wherein the cell is treated by one or more of: an irradiation, one or more of freeze and thaw cycles, a heat-shock, or a hypochlorous acid (HOCl) oxidization.

11. The method of claim 1, further comprising treating the cell by one or more of: an irradiation, one or more of freeze and thaw cycles, a heat-shock, or a hypochlorous acid (HOCl) oxidization.

12. The method of claim 1, further comprising isolating a cancer cell from the subject prior to the administration of (a) and (b).

13. The method of claim 12, further comprising irradiating the cancer cell.

14. The method of claim 1, wherein the cell is irradiated with about 10 Gray to about 1000 Gray, or about 100 Gray, or about 70 Gray prior to the administration.

15. The method of claim 1, wherein (a) and (b) are administrated in one dose or in two separate doses.

16. The method of claim 15, wherein (a) and (b) are administrated concurrently or sequentially.

17. (canceled)

18. (canceled)

19. The method of claim 1, wherein the administration is subsequent to tumor resection.

20. The method of claim 1, wherein the cancer in the subject is clinically undetectable.

21. The method of claim 1, wherein the subject is a cancer patient who has been treated by one or more of: an ablative therapy, a chemotherapy, a radiation therapy, an immune checkpoint blockade therapy, or another anti-cancer therapy.

22. The method of claim 1, further comprising treating the subject with one or more of: an ablative therapy, a chemotherapy, a radiation therapy, an immune checkpoint blockade therapy, or another anti-cancer therapy.

23-26. (canceled)

27. A vaccine composition comprising (a) an adjuvant which comprises a cowpea mosaic virus (CPMV) particle; and (b) one or more antigen(s) from a cancer or a cancer cell comprising the one or more antigen(s).

28-39. (canceled)
